SEQUENCE LISTING (2 sequences)

SEQ ID NO: 1 - DNA, 16 bp, Artificial Sequence
  Primer
acaggatcccatggcc

SEQ ID NO: 2 - DNA, 21 bp, Artificial Sequence
  Primer
ggatcgcggccgctactcgag

(19) United States
(12) Patent Application Publication
(10) Pub. No.: US 2010/0292096 A1
(43) Pub. Date: Nov. 18, 2010

(54) STRAIN AND SPECIES-SPECIFIC BORRELIA PROTEIN ARRAY

(75) Inventors: Benjamin J. Luft, Riverhead, NY (US); John F. Bruno, Islip, NY (US); Yun Xu, Stony Brook, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Research Foundation of State University of New York, Albany, NY (US)

(21) Appl. No.: 12/784,584

(22) Filed: May 21, 2010

Related U.S. Application Data
(63) Continuation-in-part of application No. PCT/US2009/002474, filed on Apr. 21, 2009.
(60) Provisional application No. 61/125,040, filed on Apr. 22, 2008.

(51) Int. Cl. C40B 30/04; G01N 33/566; C40B 40/10

(52) U.S. Cl. 506/9; 436/501; 506/18

(57) ABSTRACT

Methods of assessing a sample for the presence of antibodies to certain proteins of Borrelia burgdorferi, are described, as are methods of diagnosing Lyme disease. Microarrays of proteins of Borrelia burgdorferi are also described.

### RELATED APPLICATIONS

This application is a continuation-in-part of International Application No. PCT/US2009/002474, which designated the United States and was filed on Apr. 21, 2009, published in English, which claims the benefit of 61/125,040, filed on Apr. 22, 2008. The entire teachings of the above applications are incorporated herein by reference.

### BACKGROUND OF THE INVENTION

Lyme disease is the most common vector-borne disease in North America and Europe, and its range and incidence are increasing. Human Lyme disease is caused by several members of a group of closely related spirochetes belonging to the Borrelia burgdorferi sensu lato species complex. The spirochete is transmitted to humans via ticks of the genus Ixodes (Steere, A. C., N. Engl. J. Med. 1989; 321:586-96). It is a progressive multisystem disorder characterized by an initial cutaneous infection that can spread early in infection to secondary sites that include the nervous system, heart and joints (Masuzawa, T. et al., Microbiol. Immunol. 1996; 40:539-45; Stanek, G., Infection 1991; 19:263-7). The accurate diagnosis and treatment of Lyme disease depends on correlating objective clinical abnormalities with serological evidence of exposure to B. burgdorferi.

### SUMMARY OF THE INVENTION

The present invention is drawn to methods of assessing a test sample from an individual for antibodies to one or more proteins of Borrelia burgdorferi, such as one or more of the proteins shown in Table 1, Table 2, Table 4 or Table 5. The methods can include the use of a microarray of proteins of B. burgdorferi, such as a microarray including the proteins shown in Table 1, Table 2, Table 4 or Table 5, or subsets thereof. The invention is further drawn to methods of diagnosing Lyme disease in an individual, by assessing a test sample from the individual for antibodies to one or more proteins of B. burgdorferi, wherein the presence of the antibodies is diagnostic for disease. The invention is additionally drawn to microarrays of proteins of B. burgdorferi, such as microarrays useful in the methods.

### DETAILED DESCRIPTION OF THE INVENTION

A description of example embodiments of the invention follows.

It has been discovered that antibodies to certain cell envelope proteins are present in sera of individuals with disseminated Lyme disease. A microarray containing proteins encoded by 90 cell envelope proteins and their homologs was prepared. The microarray was exposed to sera from individuals previously diagnosed with disseminated Lyme disease. Results indicated that the sera of individuals with Lyme disease reacted with specific cell envelope proteins including those shown in Table 2. In particular, high numbers of the sera from the individuals reacted with a specific subset of those proteins—those shown in Table 1. None of the control sera from individuals without Lyme disease, reacted with the proteins of Tables 1 or 2. In addition, antibodies to certain other or additional proteins are also present in sera of individuals with Lyme disease. Results indicated that the sera of individual with Lyme disease reacted with 164 recognized proteins, shown in Table 4, and in particular with 67 highly immunogenic proteins, shown in Table 5.

As a result of this discovery, methods and microarrays are now available for the assessment of a test sample for the presence of antibodies to proteins of Borrelia. The presence of such antibodies is diagnostic for Lyme disease. In addition, methods are available to identify potential diagnostic and vaccine candidates relating to Lyme disease.

In certain methods and the microarrays of the invention, one or more cell envelope proteins are used. In certain embodiments, a set of two or more cell envelope proteins are used. Representative sets include the set of proteins shown in Table 2, and the set of proteins shown in Table 1. Other representative sets of cell envelope proteins include the set of all known and putative cell envelope proteins of B. burgdorferi. Such a set can further include homologs and paralogs of the cell envelope proteins. Other sets include sets of two or more, three or more, four or more, five or more, six or more, seven or more, eight or more, or other groups of cell envelope proteins (e.g., selected from those set forth in Table 2 or in Table 1). In one particular embodiment, the set consists essentially of the proteins set forth in Table 2. In another particular embodiment, the set consists essentially of the proteins set forth in Table 1.

In certain other methods and the microarrays of the invention, one or more other proteins as identified herein are used. In certain embodiments, a set of two or more proteins are used. Representative sets include the set of proteins shown in Table 4, and the set of proteins shown in Table 5. Other representative sets of proteins include homologs and paralogs of the listed proteins. Other sets include sets of two or more, three or more, four or more, five or more, six or more, seven or more, eight or more, or other groups of proteins (e.g., selected from those set forth in Table 4 or in Table 5). In one particular embodiment, the set consists essentially of the proteins set forth in Table 4. In another particular embodiment, the set consists essentially of the proteins set forth in Table 5.

In further methods and the microarrays of the invention, one or more other proteins as identified herein are used, other combinations of proteins are used (e.g., proteins selected from any of Tables 1, 2, 4, and 5, in combinations). For example, sets of proteins from Table 1 and Table 5, or Table 2 and Table 4, can be used in combination. Other sets include sets of two or more, three or more, four or more, five or more, six or more, seven or more, eight or more, or other groups of proteins (e.g., with one or more selected from Table 1, Table 2, Table 4 and/or Table 5).

In another embodiment of the invention, a test sample from an individual is assessed for the presence of antibodies to one or more proteins (e.g., cell envelope proteins) of B. burgdorferi The “test sample” is a sample of blood, serum, cerebrospinal fluid, or other appropriate biological fluid from the individual. In the methods, the test sample is assessed for the presence of antibodies to one or more proteins using routine methods established in the art. In one particular embodiment, the assessment is performed using a microarray of Borrelia proteins. In certain methods, for example, a microarray as described below, or a protein or set of proteins as described herein, is exposed to the test sample from the individual, and any resultant binding of antibodies (if present in the test sample) to the proteins is assessed. The presence of binding of antibodies to one or more proteins is indicative of antibodies to those proteins of B. burgdorferi. The presence of such antibodies is diagnostic for Lyme disease in the individual from whom the test sample was obtained.

The present invention also pertains to microarrays of proteins of B. burgdorferi. In one embodiment, the microarray consists essentially of all known and putative cell envelope proteins of B. burgdorferi. In another embodiment, the microarray comprises a subset of all known and putative cell envelope proteins of B. burgdorferi, such as the set the proteins set forth in Table 2. In a further embodiment, the microarray comprises a subset of the proteins set forth in Table 2 (e.g., the set of proteins set forth in Table 1). In other embodiments, other microarrays include various subsets of cell envelope proteins of B. burgdorferi, such as sets of two or more, four or more, six or more, eight or more, or other groups of cell envelope proteins as set forth in Table 2. In one particular embodiment, the microarray consists essentially of the proteins set forth in Table 2. In another particular embodiment, the microarray consists essentially of the proteins set forth in Table 1. In further embodiments, other microarrays include various subsets of proteins of B. burgdorferi, such as subsets of the proteins set forth in Table 4 or in Table 5 (e.g., sets of two or more, four or more, six or more, eight or more, or other groups of proteins as set forth in Table 4 or Table 5). In one particular embodiment, the microarray consists essentially of the proteins set forth in Table 4. In another particular embodiment, the microarray consists essentially of the proteins set forth in Table 5. Other combinations or proteins, such as combinations comprising sets of proteins from one or more of Tables 1, 2, 4 and/or 5, can also be used.

In other embodiments of the invention, methods are now available for assessment of cell surface proteins of B. burgdorferi as potential candidates for development of a diagnostic test for Lyme disease, and also for assessment of cell surface proteins of B. burgdorferi as potential candidates for development of vaccines to protect against Lyme disease. In both of these methods, one or more cell surface proteins of B. burgdorferi, such as sets of cell surface proteins as described herein (e.g., in a microarray as described above), are exposed to sera from one or more individuals known to have Lyme disease, and the proteins to which antibodies from the sera bind are then determined. For example, Cy5 intensity/Cy3 intensity ratio of fluorescence, as described in the Examples, can be used. The ratio of any proteins greater than the mean ratio of the reactivity of the Lyme sera to a negative control plus three times the standard deviation indicates significant interactions between antibodies present in the Lyme sera and the B. burgdorferi protein. Such proteins are proteins which can be used in diagnostic tests for Lyme disease (e.g., in the methods described above), as well as in microarrays as described herein, and also can be used as potential vaccine candidates.

Also, in other embodiments of the invention, methods are now available for assessment of other or additional proteins of B. burgdorferi identified herein as potential candidates for development of a diagnostic test for Lyme disease, and also for assessment of proteins of B. burgdorferi identified herein as potential candidates for development of vaccines to protect against Lyme disease. In both of these methods, one or more proteins of B. burgdorferi, such as sets of proteins as described herein in Table 4 or Table 5 (e.g., in a microarray as described above), are exposed to sera from one or more individuals known to have Lyme disease, and the proteins to which antibodies from the sera bind are then determined. For example, Cy5 intensity/Cy3 intensity ratio of fluorescence, as described in the Examples, can be used. The ratio of any proteins greater than the mean ratio of the reactivity of the Lyme sera to a negative control plus three times the standard deviation indicates significant interactions between antibodies present in the Lyme sera and the B. burgdorferi protein. Such proteins are also proteins which can be used in diagnostic tests for Lyme disease (e.g., in the methods described above), as well as in microarrays as described herein, and additionally can be used as potential vaccine candidates.

In addition, the proteins identified herein as reacting with sera of individuals with Lyme disease (e.g., those shown in Table 1 and/or Table 2 and/or Table 4 and/or Table 5) as well as the methods described herein can be used as prognostic markers enabling one skilled in the art to tailor treatment for disease by targeting those specific proteins. For example, should an individual's serum demonstrate reactivity with a particular subset of proteins, therapy can be initiated to reduce and/or eliminate the presence of those proteins in the individual, as shown by reducing and/or eliminating reactivity of the individual's serum with those proteins.

Furthermore, the proteins identified herein as reacting with sera of individuals with Lyme disease (e.g., those shown in Table 1 and/or Table 2 and/or Table 4 and/or Table 5) are useful as vaccine immunogens against Borrelia infection. Thus, the present invention is also drawn to pharmaceutical compositions which can be used to vaccinate and/or treat Borrelia infection in an animal or human. In a particular embodiment, the pharmaceutical composition comprises a Borrelia burgdorferi cell envelope protein, such as one shown in Table 1 or 2, or another Borrelia burgdorferi protein, such as one shown in Table 4 or 5, or a protein derived from such a cell envelope protein or other protein (e.g., a protein having modifications such as insertions, deletions, or other alterations, or a protein that forms part of a chimeric protein, such as those described in U.S. Pat. Nos. 6,248,562; 7,008,625; 7,060,281; and 7,179,448, the entire teachings of which are incorporated herein by reference). Combinations of the proteins described herein (e.g., those in Tables 1, 2, 4 and 5) can also be used.

The pharmaceutical composition can also be administered together with a physiologically-acceptable carrier, an excipient and/or an adjuvant. Suitable adjuvants are well known in the art (see for example PCT Publication WO 96/40290, the entire teachings of which are incorporated herein by reference), and can be used, for example, to enhance immunogenicity, potency or half-life of the proteins in the treated animal.

The pharmaceutical compositions used to vaccinate and/or treat Borrelia infection can be prepared using methods for preparing vaccines which are well known in the art. For example, the proteins described herein can be isolated and/or purified by known techniques, such as by size exclusion chromatography, affinity chromatography, ion exchange chromatography, preparative electrophoresis, selective precipitation or combinations thereof. The prepared proteins can be mixed with suitable other reagents as described herein, such that the protein is at a suitable concentration. The dosage of the protein will vary and depends upon the age, weight and/or physical condition of the animal, e.g., mammal, human, to be treated. The optimal dosage can be determined by routine optimization techniques, using suitable animal models.

Administration of the pharmaceutical composition to be used as a vaccine can be by any suitable technique. Suitable techniques for administration of the pharmaceutical composition include, but are not limited to, injection, e.g., subcutaneous injection, intramuscular injection, intravenous injection, intra peritoneal injection; mucosal administration, e.g., exposing nasal mucosa to nose drops containing the proteins of the present invention; oral administration; and DNA immunization.

The present invention is also drawn to diagnostic and/or prognostic kits which comprise the proteins described herein (e.g., in a microarray as described above). The kit also includes reagents for detecting antibody-antigen complexes that are formed between the protein and antibodies that are present in a sample, e.g., a user-supplied host sample.

### Example 1

### Cell Envelope Protein Arrays

To determine the cell envelope proteins of Borrelia burgdorferi recognized by immune sera of patients with late Lyme disease, a microarray was developed containing proteins encoded by 90 cell envelope genes and their homologs described in the annotated genomic sequence of B. burgdorferi, strain B31 (see, e.g., Fraser, C. M. et al. 1997, Nature 390(6660):580-6). (See also GenBank Accession numbers AE000789.1, AE000786.1, AE001580.1, AE001575.1, AE000790.1, AE001576.1, AE000788.1, AE000784.1, AE001578.1, AE000787.1, AE001577.1, AE000783.1, AE001582.1, AE001579.1, AE000785.1, AE000793.1, AE001581.1, AE000792.1, AE000791.1, AE000794.1, AE001583.1 and AE001584.1. The teachings of these Accession numbers are incorporated herein in their entirety.)

Materials and Methods

Serum samples. Sera were obtained from patients who participated in multicenter clinical trials conducted by the Lyme Disease Center at Stony Brook University. The serum samples were obtained singly from different subjects and all serum samples were obtained from physician-characterized patients under established guidelines with prior approval by the Committee on Research Involving Human Subjects, Stony Brook University. The samples used included a total of 13 sera from patients with late Lyme disease (Lyme arthritis or neuroborreliosis) and all tested positive for B. burgdorferi antibodies by ELISA. Normal control sera were obtained from 4 healthy donors.

Borrelia cultivation and DNA Isolation. A B. burgdorferi B31 early passage strain containing all 21 known circular and linear plasmids was used as the source of total genomic DNA (Xu Y. et al., Microb. Path. 2003; 35:269-78). Spirochetes were cultivated at 34° C. to the mid-logarithmic phase in complete Barbour-Stoenner-Kelly (BSK-H) medium. B. burgdorferi genomic DNA was isolated from late-logarithmic phase B31 by using the Qiagen Genomic-tip 500 DNA purification columns (Dunn, J. J. et al., Protein Expr. Purif. 1990; 1:159-68). In addition, B. burgdorferi isolates recovered from human patients and typed for OspC phyletic group (referred to below as OspC types) were also used in this analysis and have been described (Attie, O. et al., Infect. Gen. Evo. 2007; 7:1-12).

PCR amplification of Borrelia Lipoprotein genes. Approximately 90 ORFs encoding putative cell membrane proteins were amplified by using gene-specific primers designed from the genomic sequence of B. burgdorferi B31. Ten ng of genomic DNA was used as template in a 50-μl PCR reaction containing two ORF-specific primer pairs with different restriction sites for cloning into the T7-based expression vector pET-30 (Novagen). This vector also provides an N-terminal poly (His) affinity tag to expressed proteins to aid in purification on nickel-Sepharose columns. The 5′ primer (5′-ACAGGATCCCATGGCC+15MER ORF specific sequence) (SEQ ID NO:1) contained a NcoI site (bold). The 3′ primer (5′-GGATCGCGGCCGCTACTCGAG+15mer ORF specific) (SEQ ID NO:2) contained a NotI recognition sequence (bold). To increase the solubility properties of expressed proteins, primer sets were designed to amplify coding regions without a membrane anchoring signal sequence (Dunn, J. J. et al., Protein Expr. Purif. 1990; 1:159-68). PCR amplification was performed under stringent conditions using Platinum Taq DNA polymerase High Fidelity (Invitrogen) using conditions we have previously described (Xu Y. et al., Microb. Path. 2003; 35:269-78). The PCR products were visualized by agarose gel electrophoresis. For quantification, the products were purified (PCR purification kit, Qiagen) and quantified by fluorometry. In addition, representatives of several different OspC types were amplified as described above from human isolates that we have previously characterized (Attie, O. et al., Infect. Gen. Evo. 2007; 7:1-12). The OspC types included in this study were types A, B, C, D, E, H, I, J, K and U.

For directional cloning into the pET-30 vector, amplified products were cleaved with NcoI and NotI and inserted between the NcoI and NotI sites of pET-30 for N-terminal His-tagged proteins. Ligation reactions were transformed into E. coli GC5 competent cells and plasmids were purified using Eppendorf Perfectprep Plasmid 96 VAC Direct Bind Kit.

Protein expression and purification. Purified plasmids were transformed into E. coli BL21/DE3 competent cell for expression. Borrelia proteins containing an N-terminal poly (His) affinity tag were expressed using the Overnight Express Autoinduction protocol (Studier, W. F. et al., Protein Express. Purif. 2005; 41:207-34). Induced cells were harvested by centrifugation and resuspended in BugBuster Protein Extraction Reagent. Following clarification by centrifugation, the supernatants were saved (soluble proteins) and cell pellets were resuspended in His-binding buffer with 8M urea (insoluble proteins). Aliquots of both supernatants and pellets were run on SDS-PAGE.

N-terminal poly His-tagged proteins were purified on nickel-Sepharose columns under either native conditions (soluble proteins) or strong denaturing conditions (insoluble proteins) using RoboPop Ni-NTA His.Bind Purification Kit (Novagen). The kit is designed for filtration-based 96-well format purification of His.Tag fusion proteins.

Protein concentration was determined by the measurement of the absorbance shift when Coomassie brilliant blue G-250 reacted with protein (Bio-Rad). Protein purity was checked by SDS-PAGE.

Microarray. For microarray, proteins were printed onto nitrocellulose-coated FAST glass slides using a Microcaster 8-pin Microarray Printer. Each slide in the arrays contained 10 immobilized BSA spots for background determination and 8 immobilized His-tagged hGS2 spots, a human lipase protein, for use as a negative control. Proteome chips were probed with serum from B. burgdorferi infected patients (positive for Bb by ELISA) using the Fast Pak protein array kit. Briefly, slides were first blocked overnight at 4° C. in protein array-blocking buffer before incubation in primary Antibody (human sera and mouse anti His-Tag for quantitation) for 2 h. Antibodies were visualized with Cy5-conjugated goat anti-human IgG/IgM/IgA and Cy3-conjugated goat anti mouse IgG and the slides were stringently washed and then scanned with an Axon GenePix 4200A microarray scanner and raw data was captured and analyzed with GenePix Pro image analysis software. To minimize the variability among samples, the PMT gain was adjusted to equal 1.0 in all the arrays with power setting at 50%. A global background subtraction method was used to subtract the background from each spot using the average mean intensity value of BSA from each slide.

Data analysis. For analysis of the data generated from the arrays with human serum, the spot was considered positive and included for further ratio analysis if the median fluorescence intensity of a spot was more than 1000 and the SNR (signal-noise-ratio) of a spot was more than 4. A ratio Cy5 intensity/Cy3 intensity (protein/His-tag) for each protein was then calculated. All experiments were conducted two times, and each proteins Cy5/Cy3 ratios were averaged. The ratio of any proteins greater than the mean ratio of the reactivity of the Lyme sera to the GS2 negative control plus three times the standard deviation indicates significant interactions between antibodies present in the Lyme sera and immobilized B. burgdorferi protein.

Results and Discussion

The majority of B. burgdorferi membrane-associated proteins are lipoproteins that represent more than 8% of Borrelia's total coding capacity (Beermann, C. et al., Biochem. Biophys. Res. Commun. 2000; 267:897-905). Because of their importance as antigens and mediators of inflammation (Radolf, J. D. et al., J. Immunol. 1995; 154:2866-77) these membrane-associated proteins are of significant interest as potential vaccine targets. To identify antigens important in the human immune response to Lyme disease, a protein microarray was used to examine the serum antibody reactivity of Lyme patients with 90 Borrelia burgdorferi cell envelope proteins.

To fabricate protein microarray chips, each ORF was PCR amplified and directionally cloned into the T7 expression vector pET28b. Sequenced-confirmed plasmids were expressed using the overnight expression system, expressed proteins were purified using His resin and printed onto nitrocellulose coated FAST slides. The PCR strategy was designed to subclone a version of each membrane protein without a N-terminal signal sequence. In preliminary studies, full-length gene products appeared to be toxic when over expressed in E. coli. As a result, target proteins did not accumulate to very high levels. The truncated form of each protein lacking a signal sequence proved to be excellent over producers. (Dunn, J. J., et al., Protein Expr. Purif. 1990; 1:159-68)

When arrays were probed with sera from 13 Lyme disease patients, a considerable amount of heterogeneity was observed in reactivity of individual sera samples to the arrayed proteins (see Table 2). Of the 90 antigens, only one, BBP28, was recognized by all 13 sera samples. Three antigens, BBN39, BBO40, and BBK50, were recognized by 12 of 13 samples. Although seventy-six of the arrayed antigens were recognized by at least one sample, less than half were recognized by more than six patients. Considerable heterogeneity was also noted among arrayed proteins showing the highest seroreactivity. Of those antigens displaying the highest C5/C3 signal intensity ratios, antigens BBA25 (DbpB), BBE31 (putative P35) and BBO383 (bmpA) were recognized by less than half of the individuals. Sera from noninfected humans did not react with any of the antigens on the array (data not shown).

Although there were sample-specific responses, there was a subset of proteins recognized in common by a majority of the sera. The 25 most immunodominant antigens found in this study are presented in Table 1. Several of the 25 antigens we identified were previously reported as antigens in humans. Included are several members of the Erp gene families which code for proteins that bind to mammalian complement inhibitor factor H and Decorin-binding protein (DbpA), a borrelial surface lipoprotein that function as an adhesin promoting bacterial attachment to host cells (Casjens, S. et al., Mol Microbiol. 2000; 35, 490-516; Miller, J. C. et al., J. Clin. Microbiol. 2000; 38:1569-74; von Lackum, K. et al., Infect. Immun. 2005; 73: 7398-05; and Cinco, M. et al., FEMS Microbiol. Lett. 2000; 183:111-4). Late disseminated sera also recognized the previously established immunogens, export protein A (BBC06), P35 (BBJ41), P37 (BBK50), OspA (BBA15) and OspC (BBB19) (Fikrig, E. et al., Science; 1990:250:553-6; Funhg, B. P. et al., Infect. Immun. 1994; 62:3213-21; Champion, C. I. et al., Infect. Immun. 1994; 62: 2653-61; Aguero-Rosenfeld, M. E. et al., J. Clin. Microbiol. 1996; 34:1-9; Nowalk, A. J. et al., Infect. Immun. 2006 July; 74:3864-73).

Several members of the Borrelia gene family Pfam113 exhibited strong immunoreactivity to late disseminated human sera (Casjens, S. et al., Mol Microbiol. 2000; 35, 490-516). This lipoprotein gene family designated Mlp lipoproteins are found on both circular and linear plasmids and include BBP28, BBL28, BBO28, BBS30, BBM28 and BBN28 (Table 1). The mlp genes encode a diverse array of lipoproteins that are highly antigenic and may participate in infection processes in the mammalian host (Porcella, S. F. et al., Infect. Immun. 2000; 68: 4992-5001). Similarly, BBI42, shown to be immunogenic in a previous study with baboon sera, was highly reactive with human sera (Brooks, C. S. et al., Infect. Immun. 2006 July; 74:206-304).

To determine if the human antibody response to OspC was type specific, recombinant Osp C types A, B, C, D, E, H, I, J, K and U were generated and included as antigens in the protein array. As shown in Table 1, OspC (BBB19) was highly immunogenic in 9 of 13 sera from Lyme patients. There was no evidence found; however, of OspC type specificity in late-disseminated sera. All OspC types within a given serum sample were recognized with essentially equal signal intensities (Table 2). Among the novel, uncharacterized B. burgdorferi antigens identified in this study were BBA14 (lipoprotein), BBG23 (hypothetical protein), BBO108 (lipoprotein), BBO442 (inner membrane protein) and BBQ03 (putative outer membrane protein).

TABLE 1
Borrelia burgdorferi cell envelope proteins showing highest reactivity
to sera from patients with late disseminated Lyme
Disease as shown by protein microarray

| | Gene | | C5/C3 | # of Positive |
|---|---|---|---|---|
| Locus | Symbol | Protein Name | Ratio | Sera |
| BBP28 | mlpA | Lipoprotein | 1.8 | 13 |
| BBN39 | erpB2 | erpB2 protein | 4.6 | 12 |
| BBO40 | erpM | ErpM protein | 1.7 | 12 |
| BBK50 | — | Immunogenic protein P37, | 2.1 | 12 |
| | | putative | | |
| BBA24 | dbpA | Decorin binding protein A | 26.0 | 11 |
| BBJ09 | ospD | Outer surface protein D | 2.3 | 11 |
| BBL28 | mlpH | Lipoprotein | 1.0 | 11 |
| BBI42 | — | Outer membrane protein, | 7.6 | 10 |
| | | putative | | |
| BBQ47 | erpX | ErpX protein | 1.9 | 10 |
| BBO39 | erpL | ErpL protein | 1.2 | 10 |
| BBO28 | mlpG | Lipoprotein | 1.4 | 10 |
| BBC06 | eppA | Exported protein A | 16.2 | 9 |
| BBS41 | erpG | Outer surface protein G | 8.2 | 9 |
| BBR42 | erpY | Outer surface protein F | 5.5 | 9 |
| BBQ03 | — | Outer membrane protein, | 4.6 | 9 |
| | | putative | | |
| BBJ41 | — | Antigen, P35, putative | 4.0 | 9 |
| BBA15 | ospA | Outer surface protein A | 3.7 | 9 |
| BBB19 | ospC | Outer surface protein C | 3.9 | 9 |
| BBS30 | mlpC | Lipoprotein | 2.5 | 9 |
| BBM28 | mlpF | Lipoprotein | 1.9 | 8 |
| BBG23 | | Hypothetical protein | 1.4 | 8 |
| BBN28 | mlpI | Lipoprotein | 1.1 | 8 |
| BB0108 | | Lipoprotein | 3.1 | 7 |
| BB0442 | | Inner membrane protein | 4.8 | 7 |
| BBA14 | | Lipoprotein | 2.1 | 7 |

Table 2 indicates all of the proteins identified by serum antibodies from individuals with Lyme disease.

TABLE 2
Binding of human serum antibodies from late-disseminated Lyme
disease to B. burgdorferi proteins

| | | | | # of |
|---|---|---|---|---|
| | Gene | | C5/C3 | positive |
| Locus | Symbol | Protein name | Ratio | sera |
| BB 0028 | | Lipoprotein, putative | 3.06 | 4 |
| BB 0108 | | Basic membrane protein | 1.85 | 7 |
| BB0158 | | Antigen, S2, putative | 2.70 | 5 |
| BB0159 | | Antigen S2-related protein | 3.16 | 1 |
| BB 0213 | | Lipoprotein, putative | 1.51 | 2 |
| BB 0224 | | Lipoprotein, putative | 2.56 | 4 |
| BB 0319 | Tpn38b | Exported protein | 2.33 | 1 |
| BB0365 | | Lipoprotein LA7 | 3.57 | 6 |
| BB 0382 | bmpB | Basic membrane protein B | 1.72 | 2 |
| BB 0383 | bmpA | Basic membrane protein A | 9.33 | 5 |
| BB 0442 | | Inner membrane protein | 4.47 | 7 |
| BB 0475 | | Lipoprotein, putative | 2.00 | 6 |
| BB 0735 | rlpA | Rare lipoprotein A | 1.96 | 4 |
| BB 0758 | | Lipoprotein, putative | 5.34 | 5 |
| BB 0832 | | Lipoprotein, putative | 1.88 | 2 |
| BB A03 | | Outer membrane protein | 1.79 | 5 |
| BB A04 | | Antigen, S2 | 2.59 | 3 |
| BB A05 | | Antigen, S1 | 4.90 | 1 |
| BB A14 | | Lipoprotein, putative | 2.08 | 7 |
| BB A15 | ospA | Outer surface protein A | 3.73 | 9 |
| BB A16 | ospB | Outer surface protein B | 5.54 | 6 |
| BB A24 | dbpA | Decorin binding protein A | 25.98 | 11 |
| BB A25 | dbpB | Decorin binding protein B | 19.30 | 5 |
| BB A36 | | Lipoprotein | 0.85 | 6 |
| BB A52 | | Outer membrane protein | 0.72 | 4 |
| BB A59 | | Lipoprotein | 3.48 | 2 |
| BB A60 | | Surface lipoprotein P27 | 3.34 | 6 |
| BB A64 | | Antigen, P35 | 2.55 | 3 |
| BB A66 | | Antigen, P35, putative | 3.12 | 1 |
| BB C06 | eppA | Exported protein A | 16.15 | 9 |
| BB E09 | | Protein p23 | 0.50 | 1 |
| BB E31 | | Antigen, P35, putative | 9.95 | 5 |
| BB G22 | | Hypothetical protein | 3.86 | 6 |
| BB G23 | | Hypothetical protein | 1.22 | 8 |
| BB H32 | | Antigen, P35, putative | 4.37 | 3 |
| BB I36 | | Antigen, P35, putative | 4.70 | 7 |
| BB I42 | | Outer membrane protein, | 7.63 | 10 |
| | | putative | | |
| BB J09 | ospD | Outer surface protein D | 2.32 | 11 |
| BB J19 | | Conserved hypothetical | 20.36 | 1 |
| | | protein | | |
| BB J41 | | Antigen, P35, putative | 4.03 | 9 |
| BB K32 | | Immunogenic protein P35 | 4.30 | 3 |
| BB K45 | | Immunogenic protein P37, | 0.67 | 1 |
| | | putative | | |
| BB K48 | | Immunogenic protein P37, | 1.63 | 4 |
| | | putative | | |
| BB K50 | | Immunogenic protein P37, | 1.05 | 12 |
| | | putative | | |
| BB K52 | | Protein p23 | 1.10 | 1 |
| BB K53 | | Outer membrane protein | 6.41 | 7 |
| BB L28 | | Lipoprotein | 0.97 | 11 |
| BB M28 | Lp | Lipoprotein | 1.85 | 8 |
| BB M38 | erpK | erpK protein | 1.03 | 2 |
| BB N26 | | Outer surface protein, | 0.60 | 4 |
| | | putative | | |
| BB N28 | Lp | Lipoprotein | 1.09 | 8 |
| BB N38 | erpA | ErpA protein | 0.74 | 3 |
| BB N39 | erpB2 | erpB2 protein | 4.60 | 12 |
| BB O28 | Lp | Lipoprotein | 1.13 | 10 |
| BB O39 | erpL | erpL protein | 1.22 | 10 |
| BB O40 | erpM | erpM protein | 1.70 | 12 |
| BB P28 | | Lipoprotein | 1.72 | 13 |
| BB P38 | erpA | erpA protein | 1.35 | 6 |
| BB Q03 | | Outer membrane protein, | 4.59 | 9 |
| | | putative | | |
| BB Q04 | | Protein p23 | 1.54 | 1 |
| BB Q35 | nlpH | Congo red-binding | 0.98 | 7 |
| | | lipoprotein NlpH | | |
| BB Q47 | erpX | ErpX protein | 1.86 | 10 |
| BB R28 | Lp | Lipoprotein | 1.27 | 7 |
| BB R42 | erpY | Outer surface protein F | 5.46 | 9 |
| BB S30 | Lp | Lipoprotein | 2.45 | 9 |
| BB S41 | erpG | Outer surface protein G | 8.20 | 9 |
| OspC type C | | | 4.79 | 7 |
| OspC type E | | | 4.39 | 8 |
| OspC type J | | | 3.65 | 8 |
| OspC type I | | | 3.48 | 9 |
| OspC type K | | | 6.25 | 8 |
| OspC type U | | | 3.78 | 9 |
| OspC type B | | | 3.66 | 8 |
| OspC type D | | | 5.11 | 7 |
| OspC type H | | | 3.95 | 7 |
| OspC type A | ospC | Outer surface protein C | 2.84 | 9 |

Table 3 indicates B. Burgdorferi arrayed proteins that were negative to sera from Lyme disease patients

TABLE 3
Proteins negative to sera

| | Gene | |
|---|---|---|
| Locus | symbol | Protein name |
| BB0382 | bmpB | basic membrane protein B |
| BB0384 | bmpC | basic membrane protein C |
| BB0385 | bmpD | basic membrane protein D |
| BB0442 | | membrane-associated protein |
| BB0603 | | membrane-associated protein p66 |
| BB0758 | | lipoprotein, putative |
| BB0840 | | lipoprotein, putative |
| BBA73 | | antigen, P35, putative |
| BBB07 | | outer surface protein, putative |
| BBK37 | | immunogenic protein P37 |
| BBK46 | | immunogenic protein P37 |
| BBQ47 | | outer membrane protein |

### Example 2

### Strain-Specific Protein Array

Genome comparisons and proteomics were used to define all of the differences in gene content and gene sequence rearrangement that distinguish four Borrelia burgdorferi sensu stricto strains, B31, N40, JD1 and 297. A Borrelia microarray was then prepared containing strain specific proteins products, and a comprehensive analysis of the immune responses occurring during Borrelia infection using sera from infected individuals was prepared. It was hypothesized that specific differences in gene content revealed by this analysis which when translated into a proteomic profile would account for the dramatic differences in the human immune response to Borrelia infection that was observed in the early protein array studies (see above).

Comparative genomics of strains B31, N40, JD1 and 297. Whole-genome comparison of Borrelia isolates allowed precise measure of the evolution of gene content, gene order and gene variability, so that an optimal library of markers could be rationally selected for diagnostic as well as phylogenetic and virulence studies. The B31 genome was downloaded from the J. Craig Venter Institute Comprehensive Microbial Resource (JCVI CMR). The companion auto-annotated N40, JD1 and 297 draft sequences were also made available by the Institute for Genomic Science of the University of Maryland. The ORF sequences and their genomic coordinates were deposited into a local relational database (BORrelia Genomes or BORG) to facilitate automatic data retrieval.

BLAST searches. BLAST-P (Altschul et al., Nucl. Acids. Res. 25:3389-3402 (1997)) was run to search for each of the predicted B31, N40, JD1, and 297 ORFs (amino acid sequence). The cutoff e-value for a significant match was set at 10E-5. Significant hits were parsed using PERL scripts based on BioPerl (http://bioperl.org) and hit statistics (e-value, identity, alignment positions, etc) and then stored in the BORG database.

Overall, the searches indicated that there are very few ORFs in N40, JD1 and 297 that are not in B31. From the results of these strain comparisons, approximately 400 ORFs were assembled that could be referred to as strain specific. These included ORFs that either did not have a significant match or showed 30% or higher heterogeneity with the corresponding match from the other strains. It was possible to identify that many of these proteins had been positively selected (data not shown) Because these loci are highly variable, they were ideal candidate markers for diagnostic studies; therefore, these variable proteins were expressed and their immunogenicity was tested using protein arrays as outlined below.

Strain-specific protein arrays. A total of 416 ORFs were identified in the B. burgdorferi genome comparison. Of these, 350 (84%) ORFs produced a product that was the correct size when PCR was performed, and all 350 were successfully cloned into the T7 expression vector pET28b. Sequenced-confirmed plasmids were expressed using the overnight autoexpression system, expressed proteins were purified using IDA resin and printed onto nitrocellulose coated FAST slides. In addition, representatives of several different OspC types were amplified from human isolates. The OspC types included in this study were types A, B, C, D, E, F, G, H, I, J, K and U. Also included were the highly antigenic B31 cell envelope proteins that were identified in an earlier protein array study. (Xu, Y et al., Microb. Path.; 45: 403-407 (2008)). In total, the arrays contained approximately 400 samples including expressed Borrelia proteins, negative controls and blanks

Serum samples. Sera of patients with Lyme disease were obtained from the Centers for Disease Control (CDC) and Stony Brook University. The CDC samples included sera from 31 patients collected upon initial presentation (Samples C=0 days) and at 10 days (Samples D), 20 days (Samples E), 30 days (Samples F), 60 days (Samples G), and 90 days (Samples H) post presentation. Fourteen late Lyme samples (Late) from patients who exhibited late clinical manifestations (Lyme arthritis or neuroborreliosis) obtained from the Lyme Disease Center at Stony Brook University were also analyzed. A total of 115 samples were analyzed that included 21 Sample C, 29 Sample D, 30 Sample E, 8 Sample F, I Sample G, 12 Sample H and 14 Late samples. The Osp C genotype of all samples was determined as described (Seinost et al, 1999). Normal control sera (n=14) were obtained from healthy donors.

Results. When arrays were probed with sera from Lyme disease patients we observed a considerable amount of heterogeneity in reactivity of individual sera samples to the arrayed proteins. However, there was a subset of proteins recognized in common by a majority of the sera. Approximately 180 of the arrayed proteins detectably elicited an antibody response in humans with natural infections. Of the 180 antigens, 164 proteins were recognized by at least one of the sera samples from each time point (see Table 4). Indeed, it was discovered that all 115 samples from the various time points recognized five or more antigens from a group of 67 highly immunogenic Borrelia proteins (see Table 5). Furthermore, all sera recognized at least one of eight antigens. This uniform positivity included all 21 sera that were collected when the patient was first seen for their erythema migrans. Thus, the overall sensitivity of our Lyme assay was 100% for this initial set of sera specimens. Importantly, the sensitivity of this assay far exceeds that of the two commercially available Lyme disease assays in patients in the early stages of the disease. In addition, a BLASTx search with highly immunogenic Borrelia proteins against the NCBI non-redundant protein database excluding all recorded Borrelia sequence revealed no significant matches to other organisms.

TABLE 4
Proteins Recognized by At Least One Sera

297_cp26_0025
BB_0024
BB_0028
BB_0057
BB_0067a
BB_0108
BB_0141
BB_0158
BB_0159
BB_0167
BB_0312
BB_0319
BB_0337
BB_0345
BB_0347
BB_0382
BB_0384
BB_0385
BB_0388b
BB_0398
BB_0420c
BB_0426
BB_0432a
BB_0441
BB_0475
BB_0546
BB_0553
BB_0603
BB_0625
BB_0628
BB_0664
BB_0689
BB_0805
BB_0805a
BB_0823
BB_0832
BB_0852
BB_0858
BB_0859
BB_A04
BB_A05
BB_A14
BB_A15
BB_A16
BB_A24
BB_A25
BB_A57
BB_A60
BB_A64
BB_A65
BB_A66
BB_A68
BB_A70
BB_A73
BB_A74
BB_A76
BB_B07
BB_B09
BB_B14
BB_C05
BB_C06
BB_C10
BB_E09
BB_E19
BB_E22
BB_E31
BB_F22
BB_F23
BB_F25
BB_G22
BB_G22a
BB_G23
BB_G28
BB_G31A
BB_G32
BB_H0048
BB_H06
BB_H18
BB_H37
BB_I16A
BB_I29
BB_I36
BB_I38
BB_J08
BB_J26
BB_J28
BB_J34
BB_J36
BB_J41
BB_J45
BB_J47
BB_K0060
BB_K19
BB_K32
BB_K37
BB_K48
BB_K50
BB_K52
BB_M39
BB_P22
BB_Q03
BB_Q05
BB_Q29
BB_R22
BB_R33
BB_S22
BB_S27
BB_S30
BB_S41
BB_S42
BB_T07
Bbu297_A067.5
Bbu297_F32
Bbu297_F33
Bbu297_F34
Bbu297_J05
Bbu297_O28
Bbu297_O29a
Bbu297_P39
Bbu297_R41
Bbu297_W37
Bbu297_W37a
Bbu297_W44
Bbu297_X22
Bbu297_Y03
Bbu297_Y14
BbuJD1_AA09
BbuJD1_F28
BbuJD1_F29
BbuJD1_F31
BbuJD1_H43
BbuJD1_J05
BbuJD1_L39
BbuJD1_PV21
BbuJD1_PV46
BbuJD1_S13
BbuJD1_Z01a
BbuJD1_Z02
BbuJD1_Z32
BbuN40_D04
BbuN40_G20
BbuN40_G20A
BbuN40_I02
BbuN40_R06
BbuN40_V37
BbuN40_V38
BbuN40_Y06
BbuN40_Y07
BbuN40_Y09
BbuN40_Y14a
JD1_main_0909
ORFZ10200
ORFZ10236
ORFZ10239
ospC A
ospC B1
ospC C
ospC E
ospC F
ospC H
ospC I
ospC J
ospC K
ospC U

TABLE 5
Highly Immunogenic Proteins Recognized by Multiple Sera

| | C = 0 | D = 10 | E = 20 | F = 30 | G = 60 | H = 90 | | |
|---|---|---|---|---|---|---|---|---|
| | day | day | day | day | day | day | Late | Chronic |
| Locus | (n = 21) | (n = 29) | (n = 30) | (n = 8) | (n = 1) | (n = 12) | (n = 14) | (n = 33) |
| BB_0024 | | | 8 | 1 | | | | |
| BB_0028 | 8 | 9 | 7 | 6 | 1 | 6 | | 3 |
| BB_0057 | 3 | 6 | 3 | 2 | | 3 | 3 | |
| BB_0108 | 7 | 6 | 4 | 3 | 1 | 9 | 1 | 3 |
| BB_0141 | 5 | 9 | 5 | 3 | | 7 | 3 | 4 |
| BB_0312 | 8 | 9 | 9 | 6 | | 6 | 1 | 5 |
| BB_0345 | 5 | 8 | 6 | 6 | | 6 | 2 | 2 |
| BB_0347 | 4 | 4 | 4 | 3 | | 6 | 5 | 11 |
| BB_0385 | 3 | 12 | 6 | 3 | | 5 | | 3 |
| BB_0388b | 3 | 12 | 8 | 6 | | 6 | 2 | 3 |
| BB_0441 | | 5 | 2 | | | 3 | | 2 |
| BB_0475 | 4 | 4 | | 1 | | 4 | 3 | 3 |
| BB_0603 | 6 | 8 | 9 | 2 | | 4 | 4 | 8 |
| BB_0625 | 1 | 10 | 7 | 2 | | 3 | | |
| BB_0628 | 2 | 4 | 3 | 1 | | 2 | | |
| BB_0689 | 2 | 2 | 1 | 2 | | 4 | | |
| BB_0805 | 7 | 9 | 7 | 5 | | 3 | 2 | 4 |
| BB_0823 | 1 | | 1 | | | | | |
| BB_0852 | 3 | 2 | 3 | 5 | | 4 | 2 | 3 |
| BB_0858 | 5 | 4 | 4 | 4 | | 5 | 2 | 3 |
| BB_A04 | 4 | 6 | 5 | 2 | | 1 | 3 | 3 |
| BB_A05 | 6 | 8 | 7 | 4 | | 7 | | 3 |
| BB_A15 | 6 | 6 | 5 | 5 | | 7 | 4 | 6 |
| BB_A24 | | | | | | | 8 | 23 |
| BB_A25 | | | | | | | 9 | 15 |
| BB_A57 | 1 | | 2 | | | 1 | | |
| BB_A66 | 2 | 6 | 4 | 3 | | 5 | 4 | 2 |
| BB_A68 | 4 | 5 | 3 | 4 | | 1 | 3 | 3 |
| BB_A70 | | 6 | 5 | | | 3 | | 2 |
| BB_B14 | 1 | 6 | 6 | 2 | | 5 | | |
| BB_C05 | 5 | 9 | 11 | 4 | | 6 | 2 | 3 |
| BB_C06 | 1 | | | | | | 7 | 1 |
| BB_E09 | 9 | 7 | 8 | 6 | | 9 | 2 | 4 |
| BB_E31 | 3 | 5 | 5 | 1 | | 2 | | 1 |
| BB_G22 | 4 | 11 | 12 | 6 | | 9 | 4 | 6 |
| BB_G32 | 3 | 8 | 3 | 2 | | 4 | 5 | |
| BB_H06 | 7 | 14 | 12 | 2 | | 7 | | 4 |
| BB_J08 | 3 | | 3 | | | 1 | | |
| BB_J34 | 3 | 2 | 2 | 2 | | 3 | 1 | 2 |
| BB_J41 | 1 | 7 | 1 | 3 | | 4 | 2 | |
| BB_J47 | 3 | 5 | 4 | 2 | | 4 | 2 | 3 |
| BB_K0060 | 10 | 9 | 9 | 5 | | 6 | | 2 |
| BB_K19 | 1 | 6 | 1 | | | 3 | 5 | 17 |
| BB_K37 | 4 | 3 | 1 | 4 | | 4 | | 2 |
| BB_M39 | 2 | 4 | 3 | 2 | | 3 | | |
| BB_Q03 | 5 | 7 | 8 | 5 | | 7 | 9 | 4 |
| BB_S27 | 5 | 9 | 9 | 5 | | 6 | | 3 |
| BB_S41 | 4 | 7 | 5 | 4 | | 6 | 2 | 2 |
| BB_S42 | 2 | 6 | 6 | 3 | | | 3 | 3 |
| Bbu297_F33 | 1 | 10 | 8 | 2 | | 1 | 9 | 21 |
| Bbu297_F34 | 1 | 10 | 7 | 1 | | 1 | 6 | 11 |
| Bbu297_J05 | 2 | 3 | 1 | 2 | | 3 | 1 | |
| Bbu297_P39 | | | | | | | 1 | 9 |
| Bbu297_W37 | 3 | | | | | 4 | | 2 |
| Bbu297_W44 | 5 | 8 | 9 | 5 | | 8 | | 2 |
| BbuJD1_AA09 | 6 | 7 | 7 | 5 | | 7 | 2 | 4 |
| BbuJD1_F29 | 2 | 10 | 8 | | | | 8 | 2 |
| BbuJD1_139 | 1 | 4 | 9 | 1 | | 3 | 2 | 3 |
| BbuJD1_PV21 | 3 | 8 | 8 | 4 | | 6 | | |
| BbuJD1_Z01a | 6 | 15 | 5 | 3 | | 7 | 9 | 5 |
| BbuJD1_Z02 | | | | | | | 3 | |
| BbuN40_V38 | 2 | 10 | 7 | 3 | | 5 | | |
| BbuN40_Y07 | 1 | 6 | 5 | 1 | | | | |
| JD1_main_0909 | 2 | 10 | 7 | 3 | | 6 | 2 | |
| ORFZ10200 | 2 | 7 | 2 | 1 | | 3 | | 3 |
| ORFZ10236 | 9 | 8 | 6 | 3 | | 5 | 5 | 4 |
| ospCs | 2 | 14 | 12 | 3 | | 4 | 5 | 4 |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method for assessing a test sample from an individual for the presence of antibodies to one or more cell envelope proteins of Borrelia burgdorferi, comprising exposing the test sample to one or more proteins selected from the group consisting of the proteins shown in Table 1, and assessing the test sample for binding of antibodies to said protein(s).

2. The method of claim 1, wherein the test sample from the individual is assessed for the presence of antibodies to each of the proteins shown in Table 1.

3. The method of claim 1, wherein the assessment of the test sample includes the use of a microarray comprising the protein(s).

4. The method of claim 1, wherein the microarray comprises all of the proteins shown in Table 1.

5. A method for assessing a test sample from an individual for the presence of antibodies to one or more cell envelope proteins of Borrelia burgdorferi, comprising exposing the test sample to one or more proteins selected from the group consisting of the proteins shown in Table 2, and assessing the test sample for binding of antibodies to said protein(s).

6. The method of claim 5, wherein the test sample from the individual is assessed for the presence of antibodies to each of the proteins shown in Table 2.

7. The method of claim 5, wherein the assessment of the test sample includes the use of a microarray comprising the protein(s).

8. The method of claim 5, wherein the microarray comprises all of the proteins shown in Table 2.

9. A method for diagnosing Lyme disease in an individual, comprising assessing a test sample from the individual for the presence of antibodies to one or more cell envelope proteins, wherein the presence of said antibodies is diagnostic for Lyme disease.

10. The method of claim 9, wherein a cell envelope protein is selected from the group consisting of the proteins shown in Table 1.

11. The method of claim 9, wherein a cell envelope protein is selected from the group consisting of the proteins shown in Table 2.

12. The method of claim 9, wherein the test sample from the individual is assessed for the presence of antibodies to at least two cell envelope proteins selected from the group consisting of the proteins shown in Table 2.

13. The method of claim 9, wherein the test sample from the individual is assessed for the presence of antibodies to at least four cell envelope proteins selected from the group consisting of the proteins shown in Table 2.

14. The method of claim 9, wherein the test sample from the individual is assessed for the presence of antibodies to each of the proteins shown in Table 1.

15. The method of claim 9, wherein the test sample from the individual is assessed for the presence of antibodies to each of the proteins shown in Table 2.

16. The method of claim 9, wherein the assessment of the test sample includes the use of a microarray comprising the protein(s).

17. The method of claim 1, wherein at least one protein is a protein with an open reading frame at a locus selected from the group consisting of: BBP28, BBN39, BBO40 and BBK40.

18. The method of claim 1, wherein at least one protein is a protein with an open reading frame at a locus selected from the group consisting of: BBA25, BBE31, and BBO383.

19. The method of claim 16, wherein the microarray comprises all of the proteins shown in Table 1.

20. The method of claim 16, wherein the microarray comprises all of the proteins shown in Table 2.

21. A microarray comprising the cell envelope proteins shown in Table 1.

22. A microarray comprising the cell envelope proteins shown in Table 2.

23. A method for assessing a test sample from an individual for the presence of antibodies to one or more proteins of Borrelia burgdorferi, comprising exposing the test sample to one or more proteins selected from the group consisting of the proteins shown in Table 5, and assessing the test sample for binding of antibodies to said protein(s).

24. The method of claim 23, wherein the test sample from the individual is assessed for the presence of antibodies to each of the proteins shown in Table 5.

25. The method of claim 23, wherein the assessment of the test sample includes the use of a microarray comprising the protein(s).

26. The method of claim 23, wherein the microarray comprises all of the proteins shown in Table 5.

27. A method for assessing a test sample from an individual for the presence of antibodies to one or more proteins of Borrelia burgdorferi, comprising exposing the test sample to one or more proteins selected from the group consisting of the proteins shown in Table 4, and assessing the test sample for binding of antibodies to said protein(s).

28. The method of claim 27, wherein the test sample from the individual is assessed for the presence of antibodies to each of the proteins shown in Table 4.

29. The method of claim 27, wherein the assessment of the test sample includes the use of a microarray comprising the protein(s).

30. The method of claim 27, wherein the microarray comprises all of the proteins shown in Table 4.

31. A method for diagnosing Lyme disease in an individual, comprising assessing a test sample from the individual for the presence of antibodies to one or more proteins selected from the group consisting of the proteins shown in Table 5, wherein the presence of said antibodies is diagnostic for Lyme disease.

32. The method of claim 31, wherein the test sample from the individual is assessed for the presence of antibodies to each of the proteins shown in Table 5.

33. The method of claim 31, wherein the assessment of the test sample includes the use of a microarray comprising the protein(s).

34. A method for diagnosing Lyme disease in an individual, comprising assessing a test sample from the individual for the presence of antibodies to one or more proteins selected from the group consisting of the proteins shown in Table 4, wherein the presence of said antibodies is diagnostic for Lyme disease.

35. The method of claim 34, wherein the test sample from the individual is assessed for the presence of antibodies to each of the proteins shown in Table 4.

36. The method of claim 34, wherein the assessment of the test sample includes the use of a microarray comprising the protein(s).

37. A microarray comprising the cell envelope proteins shown in Table 4.

38. A microarray comprising the cell envelope proteins shown in Table 5.
